(19) United States
(12) Patent Application Publication
(10) Pub. No.: US 2019/0282729 A1
(43) Pub. Date: Sep. 19, 2019

(54) FOAMING ACTION PHARMACEUTICAL HEMOSTAT

(71) Applicant: Joshua R&D Technologies, LLC, Plano, TX (US)

(72) Inventor: David Pickett, Plano, TX (US)

(21) Appl. No.: 16/429,050

(22) Filed: Jun. 2, 2019

Related U.S. Application Data
(63) Continuation-in-part of application No. 15/150,094, filed on May 9, 2016, now abandoned.

(51) Int. Cl. A61L 26/00; A61K 9/70; A61F 13/26; A61F 13/38; A61M 3/02

(52) U.S. Cl. CPC A61L 26/0057; A61K 9/7007; A61F 2013/00642; A61F 13/38; A61M 3/0283; A61F 13/266

(57) ABSTRACT

A dissolvable foaming action hemostat may be compressed and may fully dissolve in a wound. The foaming action hemostat may use a liquid gel, a dried gel, a dry powder, a shape, or a solid “plug” like medium that may resemble a sponge or a sponge-like material and may include a foaming agent to spread therapeutic ingredients throughout the wound to control or stop bleeding. The foaming action hemostat also may introduce pharmaceuticals and/or other compounds into a wound to control/mitigate shock, calm the patient, administer antibiotics, administer anti-sepsis compounds, control infection, control sepsis, and/or mitigate pain. No harmful foreign matter is introduced in using a foaming action hemostat. This eliminates the need to surgically remove introduced foreign matter later. Use of a foaming action hemostat also may reduce the likelihood of trauma that sometimes occurs when foreign matter is left in place inside a wound.

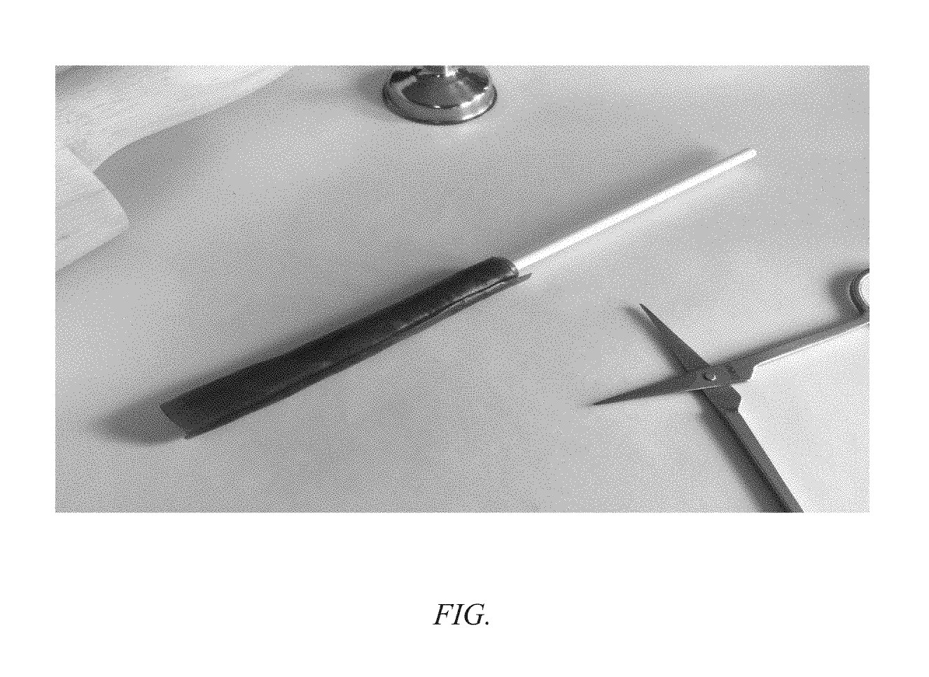

### CROSS-REFERENCE TO RELATED APPLICATION

This application is a continuation-in-part of U.S. application Ser. No. 15/150,094 filed May 9, 2016, entitled “Chemically Foaming Hemostat,” which is incorporated herein by reference in its entirety.

### FIELD OF THE DISCLOSURE

The present disclosure generally relates to hemostats, and more particularly, to a foaming action pharmaceutical dissolvable hemostat, including sponge form.

### BACKGROUND

A major cause of death from accidents and war is blood loss from uncontrolled bleeding, such as may be suffered from a wound where the patient loses so much blood that his/her body can no longer function. Not all such blood loss is rapid; an unconscious, disabled, unaware, helpless, and/or unattended victim may even bleed to death from a relatively slow, continuous, unabated flow. Internal soft tissue injuries that may not be outwardly visible to medical personnel are also a leading cause of death due to blood loss or “bleeding out.”

When there is bleeding from puncture/tear wounds or burns, tissues associated with the wound can be exposed to bacteria, viruses, mold, spores, fungi and other contaminants. This contamination—which may result in sepsis, infection, inflammation and/or swelling—can be controlled through use of antiseptics such as alcohols, quaternary ammonium compounds, chlorhexidine gluconate, hydrogen peroxide, nano-silver, and through pharmaceuticals that may kill pathogens. Tissues where pathogens are not treated/killed are highly susceptible to infections, requiring the introduction of antibiotics. Uncontrolled infections can cause tissue death, resulting in organ death and infection of multiple organs and systems, resulting in patient disabilities and/or death.

Presently, the primary means for a first responder or other medical personnel to stop blood loss is the use of pressure, a or mechanical hemostat instrument/tool to pinch off the bleeders, and/or a tourniquet, and/or a field dressing or gauze pad pressed firmly against the wound to close/pinch off the artery or vein from which blood flows. These methodologies may slow blood flow, which can encourage clotting, until repair surgery can be performed. Unfortunately, at the same time, there also may be blood clotting into the bandages, dressings, sutures, packing, sponges, and other foreign matter into the wound, often requiring even more extensive surgery to remove these harmful, foreign materials from the wound and can cause even more blood loss, stress, and shock to the patient. Also, if such harmful foreign matter, such as bandages, gauze, dressings, sponges, etc. is not completely removed from a wound, infections, cysts, and even disability or death may occur.

Surgery may permanently stop the blood loss, but often there is a long time period between the injury incident and repair surgery. A first responder or other medical personnel may have expertise at stopping bleeding, but still may not be able to stop massive bleeding, bleeding from numerous wounds or, more typically, wounds positioned such that application of pressure does not pinch off the “bleeders,” and/or the injuries or wounds may be so profound that arteries or veins are not available to pinch off. Further, first responders' mechanical hemostats may not be removed from wounds without surgical techniques.

Presently, first responders may use various chemicals and pharmaceuticals to cause wound clotting, prevent sepsis, mitigate pain, etc. Such current methods are accomplished by dusting powder onto the wound (not throughout the wound), using field dressings coated with clotting agents (again not throughout the wound), or introduction of chemically treated sponges. These methods do not treat all the surfaces in the wound and leave harmful foreign matter for the surgeon to remove later, causing new trauma, bleeding, and patient trauma/shock.

### SUMMARY

Upon contact with the water in the blood, a foaming action pharmaceutical hemostat according to embodiments of the present disclosure uses a vigorous foaming action to flow and spread the included treatment pharmaceuticals and chemicals deep into and throughout the wound tissues while leaving no harmful foreign matter in the wound. Embodiments of the present disclosure may provide a foaming action pharmaceutical hemostat, comprising a gel or material, in sheets, formed shapes, a liquid, or a dried powder or fully dissolvable sheet or shapes of material provided to form the fully dissoluble foaming action pharmaceutical hemostat. The foaming action pharmaceutical hemostat may be an anhydrous gel or material applied to a wound in sheets, liquid, a dried powder or formed gel shapes and sheets, that completely dissolve in the wound and leave no harmful residue in the wound. The foaming action pharmaceutical hemostat material may be compressed, and remain compressed, in the wound until the hemostat is fully dissolved. The fully dissolvable material may be pre-formed and may be made by made by drying a material that may be permeated with gas bubbles to resemble a sponge structure or as non-sponge forms including sheets, liquid, or dried powder. The fully dissolvable material may be in the form of a field dressing, or coated onto or impregnated into a dressing. The foaming action pharmaceutical hemostat may include a foaming agent that may be provided in the sheet or the shapes of material. The sheet, shape, liquid, or powder of material may be permeated with pharmaceuticals and other ingredients and, through foaming action, will expand to flow and to permeate all tissues throughout the wound, fully coating all the tissues with the included therapeutic chemicals and pharmaceuticals. The fully dissoluble foaming action pharmaceutical gel or material may include an insertion mechanism. The insertion mechanism may be a sterile stick or tube or wire or a syringe for dispensing liquids or shapes and may further include a cover shield tube for covering the sterile stick or wire. When the fully dissoluble foaming action pharmaceutical gel or sponge form hemostat is inserted into the wound, the sterile stick may hold a foaming agent in place, and the optional cover shield tube may then be withdrawn. The fully dissoluble foaming action pharmaceutical gel or material, or sponge form hemostat, may further include dyes, radiation markers, radioactive markers, x-ray markers, one or more sensors, detectors or wires to monitor the wound, and/or one or more tubes that may stay in the wound for sensing and to evacuate and/or introduce a solid or liquid or gas into the wound.

Other embodiments of the present disclosure may provide a sponge form of the foaming action hemostat that may include a foaming gel or material formed into a fully dissolvable sheet or small shapes or shapes of material that resemble a sponge in structure. The sponge foaming action hemostat may include a first mixture formed from mixing an anhydrous gelling agent with a sterile anhydrous liquid. The mixture may further include one or more additional anhydrous compounds selected from the group comprising shock mitigation agents, antibiotics, nano-silver particles, pain relieving agents, anti-sepsis agents and other selected agents, and pharmaceuticals that may be desirable to place into the wound. Chemicals and pharmaceuticals may be added to the first mixture and blended to form the foaming anhydrous gel material of the foaming action hemostat, wherein upon coming into contact with the water in the blood in a wound, the foaming gel or liquid or powder hemostat may aggressively foam, flow and expand throughout the wound, and bleeding in the wound may abate due to the pressure of the foaming action and the chemicals and pharmaceuticals that may be present in the foam, permeating and treating the entire tissue structures throughout the wound. Embodiments of the present disclosure may provide a foaming action pharmaceutical hemostat dissolvable sponge form hemostat comprising a fully dissolvable gel or material in sheets or formed shapes of material provided to form a fully dissoluble foaming action pharmaceutical sponge form of the hemostat. The foaming action pharmaceutical gel or other material hemostat may be applied to a wound or burn in sheets, liquid, powder or other shapes, and fully dissolve in the wound, leaving no harmful residue in the wound. The sponge form of the foaming action hemostat may be compressed and remain compressed in the wound until the sponge form foaming action hemostat is fully dissolved. The fully dissolvable material may be pre-foamed and may be made by drying a gel, or material that may be permeated with gas bubbles, to resemble a compressible sponge. The fully dissolvable sponge material may be in the form of a field dressing or shapes or sheets. The sponge foaming action hemostat may include a foaming agent that may be provided in the sheet or the shapes of material. The sheet or shapes of material may be permeated with pharmaceuticals and other ingredients and, through foaming action, will expand and flow to permeate all tissues throughout the wound, with the included therapeutic chemicals and pharmaceuticals. The fully dissolvable foaming action pharmaceutical sponge form of hemostat may include an insertion mechanism. The insertion mechanism may be a sterile stick or tube or wire or a syringe and may further include a cover shield tube covering the sterile stick or wire, wherein upon insertion of the fully dissolvable foaming action pharmaceutical sponge hemostat into the wound, the sterile stick may hold a foaming agent in place and the optional cover shield tube may then be withdrawn. The fully water-in-the-blood dissoluble foaming action pharmaceutical sponge form hemostat may further include a dissolvable or non-dissolvable radiation markers, radioactive markers, x-ray markers, one or more sensors, detectors or wires to monitor the wound, and/or one or more tubes that may stay in the wound for sensing and to evacuate and/or introduce a solid or liquid into the wound.

Embodiments of the present disclosure may provide a foaming action pharmaceutical hemostat, comprising: at least one fully dissolvable sheet of material provided to form the foaming action pharmaceutical hemostat, wherein the foaming action pharmaceutical hemostat is arranged in a wound, foams vigorously in the wound, fully dissolves in the wound, and leaves no harmful residue in the wound. The foaming action pharmaceutical hemostat may be compressed and remain compressed in the wound until fully dissolved. The at least one fully dissolvable sheet of material may be pre-foamed and dry with gas bubbles to resemble a sponge structure. The at least one fully dissolvable sheet of material may be provided in the form of a field dressing. Upon foaming action, the at least one fully dissolvable sheet of material may permeate pharmaceuticals throughout the wound. The foaming action hemostat also may include an insertion mechanism, wherein the insertion mechanism may be a sterile stick or tube or wire. The insertion mechanism may also include a cover shield tube covering the sterile stick or tube or wire, wherein upon insertion of the foaming action pharmaceutical hemostat into the wound, the sterile stick or tube or wire may hold the foaming action pharmaceutical hemostat in place and the cover shield tube may be withdrawn. The foaming action hemostat also may include a radiation or x-ray marker or other material that can be detected and disclose the location of the material throughout the wound. The foaming action hemostat also may include one or more sensors, detectors or wires to monitor the wound. The foaming action hemostat also may include one or more tubes to evacuate and introduce a solid or liquid into the wound.

Further embodiments of the present disclosure may include a foaming action hemostat comprising: a fully dissolvable sheet or piece of material; and a foaming gel comprising: a first mixture formed from mixing an anhydrous gelling agent with a sterile anhydrous liquid; and a foaming agent and a pharmaceutical-type clotting agent added to the first mixture and blended to form the foaming gel, wherein the foaming gel may be arranged to aggressively foam, expand throughout a wound, until bleeding in the wound abates, wherein the foaming action hemostat may be arranged in the wound, fully dissolve in the wound, and leave no harmful residue in the wound. The foaming gel also may include one or more additional compounds selected from the group comprising: shock mitigation agents, antibiotics, nano-silver particles, pain relieving agents, and anti-sepsis agents. A sterile stick or wire may be provided to arrange the foaming action hemostat in the wound. The foaming action hemostat may remain compressed in the wound until the foaming action hemostat fully dissolves. The foaming action hemostat may be water and therefore blood-soluble. The foaming action hemostat may automatically begin to dissolve and foam immediately after insertion into the wound and contacting with the water in the blood. The foaming action hemostat may fully dissolve and stop foaming after a predetermined period of time. The foaming action hemostat may permeate pharmaceuticals and other ingredients throughout the wound.

Other embodiments of the present disclosure may provide a hemostatic apparatus, the apparatus comprising: a fully dissolvable foaming action hemostat including a foaming agent, wherein the fully dissolvable foaming action hemostat may be introduced into a wound and fully dissolve in the wound after foaming; and a sterile stick or wire may be provided to introduce the foaming action hemostat into the wound, wherein the foaming action hemostat may take a form selected from the group comprising: a sheet, a shape, a liquid, a powder, or small cubes, and wherein upon contact with the water in the blood, the foaming agent may activate to foam in the wound and bleeding associated with the wound may be controlled or stopped. The foaming agent may be arranged to impregnate the wound with one or more compounds selected from the group comprising: clotting agents, shock mitigation agents, antibiotics, nano-silver particles, pain relieving agents, and anti-sepsis agents, wherein activation of the foaming agent in the wound introduces the one or more compounds into the wound.

Other technical features may be readily apparent to one skilled in the art from the following figures, descriptions and claims.

### BRIEF DESCRIPTION OF THE DRAWING

For a more complete understanding of this disclosure, reference is now made to the following description, taken in conjunction with the accompanying drawing, in which:

The figure depicts a hemostat according to an embodiment of the present disclosure.

### DETAILED DESCRIPTION

Embodiments of the present disclosure relate to hemostasis and control of bleeding that lead, even in complex wounds and in the presence of contaminants, to faster wound healing. In hemostasis, a wound may be sealed until the tissues can be repaired. Using a water in the blood dissolvable foaming action hemostat according to embodiments of the present disclosure may control bleeding while leaving no harmful foreign matter residue and also may allow for introduction of pharmaceuticals and/or other compounds throughout the wound and/or may include sensors that may be left in the wound to further treat and monitor a patient and tubes to evacuate and add materials. The foaming action hemostat may eliminate the need for surgery to remove harmful foreign matter from a wound, and may eliminate shock, trauma, sepsis, infection, and/or bleeding that may be caused by removing harmful foreign matter from a wound. A hemostat, according to embodiments of the present disclosure, may be compressed and may be made of a fully dissolvable material such as a pre-foamed gel sheet, liquid, dried powder and shape including those that may appear to be a sponge. It should be appreciated that the hemostat may fully expand and foam, flowing the therapeutic chemicals and pharmaceuticals throughout the wound, and then dissolve in a matter of minutes. The hemostat may be compressed or in a compressed state before entering the wound and while in the wound. The fully dissolvable foaming action hemostat material may be arranged inside of a wound and may prevent the wound from being contaminated with harmful foreign matter and/or by the hemostat itself or particles thereof. The foaming action hemostat will leave no harmful residue inside of the wound. The hemostat may be saturated by blood in a wound, begin foaming into an expanding foam that will not harm blood, and the foaming may expand and flow and spread to every tissue throughout the wound.

A dissolvable foaming action hemostat, according to embodiments of the present disclosure, may be made in the form of a sponge. The dissolvable foaming action sponge hemostat may be compressed and fully dissolve and foam inside of wounds. The dissolvable foaming action hemostat, such as in a sponge form, may be placed in wounds and may fully dissolve, foam and expand, leaving no harmful foreign matter It should be appreciated that the dissolvable foaming action hemostat may be blood-soluble with the water in the blood, initiating the foaming action. It should further be appreciated that the dissolvable foaming action hemostat may be made of a blood-tolerant dried gel that may include a vegetable, animal, or chemical material. The dissolvable foaming action hemostat may be applied to dressings, affixed to dressings, impregnated into dressings, coated onto dressings, and/or dipped in dressings. The foaming action hemostat leaves no harmful foreign matter in wounds in embodiments of the present disclosure.

A dissolvable foaming action hemostat according to embodiments of the present disclosure may be chemically foaming and may utilize a foaming gel, powder, solid and/or liquid, that may be in the form of a liquid gel, a dried gel, a powder, a solid shape, a sponge, and/or a liquid that may foam when placed in contact with the water in the blood, leaving no harmful foreign matter in the wound. A dissolvable foaming action sponge hemostat may foam when placed in contact with the water in the blood in the wound in some embodiments of the present disclosure. The dissolvable foaming action sponge hemostat may be in the form of sponge sheets that may be separated or cut into sponge shapes, and the dissolvable foaming action sponge hemostat may be made of a chemically foaming hemostat gel or material. It should be appreciated that the dissolvable foaming action sponge hemostat may be a sheet or shape that may be pre-formed and may be cut to resemble a sponge or other shapes without departing from the present disclosure. It should further be appreciated that the dissolvable foaming action sponge hemostat may contain a gas that may provide bubbles in the hemostat shape and may cause the foaming action hemostat to appear to be a sponge. This foaming action will expand and flow throughout the wound, and stop bleeding associated with the wound in even the most grievous and deep wounds until surgical treatment may be administered (which may sometimes be several hours). The foaming action in combination with the pressure associated with applying a wound dressing and/or manually applied pressure may cause the foam to increase its pressure within the wound and therefore, to push the pharmaceuticals and other included agents throughout the damaged tissues, arteries and veins, where the agents will be in contact with the injured tissues. The foam pressure may help or equal or exceed the blood's pressure, further stopping the flow of blood and further pressing the chemical and pharmaceutical agents into the damaged tissues. The foaming gel or powder or liquid also may be used as a carrier for one or more pharmaceuticals or other compounds that may distributed throughout the wound. The foaming action's pressure may block entry of contaminants into the wound and damaged tissues. It should be appreciated that the dissolvable hemostat foaming action may block entry of harmful foreign matter into the wound and may provide foaming action that drives the pharmaceuticals within the foaming gel throughout the entire wound.

A foaming action hemostat according to embodiments of the present disclosure may also stop internal bleeding, when the location of the injury can be reasonably approximated. An incision may be made in the location, and then the bleeding area may be packed with chemically foaming dissolvable hemostat(s). The patient may be stabilized long-term, and the triage level may be lowered. While the hemostat may be in the form of bandages, sponges, dressing, and/or tapes, no harmful foreign matter or materials (i.e., non-hemostatic bandages, sponges, dressings, tapes, and additional materials) need be introduced in connection with use of a hemostat according to embodiments of the present disclosure. This eliminates the need to surgically remove harmful foreign matter at a later time. It also reduces the likelihood of trauma and infection that sometimes occurs when harmful foreign matter is left in place inside a wound. The reduction of surgical time to remove harmful foreign matter may prevent the patient from dying in surgery or afterward from shock and trauma and other conditions caused by long surgical procedures, including reduced loss of the patient's natural blood.

As described in more detail herein, a foaming action hemostat, according to some embodiments of the present disclosure, may operate in a manner similar to a tampon in that a plug formed of a foaming gel may be inserted into a wound using a sterile insertion stick. The foaming action hemostat may automatically begin to fully foam and dissolve in the wound immediately after insertion into the wound. The plug in the foaming action hemostat may be impregnated with pharmaceuticals and/or other compounds that may be used to stop bleeding, control/mitigate shock, calm the patient, administer antibiotics, administer anti-sepsis compounds, control infection, control sepsis, and mitigate pain. However, other types of pharmaceuticals and/or compounds may be introduced to address other issues without departing from the present disclosure.

A foaming action hemostat, according to embodiments of the present disclosure, may include a dissolvable sponge-like material, a foaming gel, a liquid, a solid shape, and/or a powder that, when activated by contact with the water in the blood, introduces clotting pharmaceuticals and/or other compounds and pharmaceuticals into the wound. The foaming action hemostat may be uniformly applied directly to the wound and/or leaking blood vessels.

In an embodiment of the present disclosure, the foaming action gel may be formed by mixing prescription-grade collagen powder (or a similar gelling agent) with a sterile anhydrous liquid, such as anhydrous ethyl alcohol, or a light evaporative anhydrous oil that may dry/flash off in a freeze-dry process or in a low temperature oven. This mixing step may be performed under a sterile laminar flow air hood or another similar environment. Once mixed, the water/blood-activated foaming agent may then be added along with a pharmaceutical-type clotting agent. The anhydrous foaming agent does not result in foaming of the foaming action gel until it comes in contact with water in the blood. While various foaming agents may be used without departing from the present disclosure, anhydrous sodium bicarbonate may be utilized in an embodiment of the present disclosure with an activator such as anhydrous acidic acid crystals. Upon foaming, presence of the foaming agent in the wound may enable a uniform, even spreading of pharmaceuticals or other compounds flowing throughout the wound, and the foaming action may apply pressure to the wound that may also resist/stop blood flow. The pharmaceutical hemostat according to embodiments of the present disclosure may be prepared as a powder or a liquid with no gel added, so that the foaming action liquid or powder can be dusted or poured onto or into the wound and the liquid may be sprayed or squirted or injected onto or into the wound as desired.

In some embodiments of the present disclosure, other compounds may be added at the time that the pharmaceutical-type clotting agent is added, including, but not limited to, shock mitigation agents, antibiotics, nano-silver particles at approximately 50-30 nm (to kill viruses and bacteria), pain relieving agents such as topical xylocaine, or other pharmaceuticals or additives. Each of these compounds will be described in more detail below.

Upon addition of one or more of these compounds, the resultant foaming action gel may be blended until it is stiff in form and then rolled into one or more thin sheets, or it may be formed into a solid shape and dried to a stiff, flexible consistency, not brittle which might break in handling or use. In embodiments of the present disclosure, the gel may then be freeze-dried, vacuum dried, or heated in a warm/hot air oven until the sheets can be rolled up tightly. The gel sheets may be rolled (i.e., using a sterile non-stick roller) tightly onto a sterile stick or tube or wire until in the shape/thickness desired for insertion into the wound and trimmed to size (see, e.g., figure). The trimmings may then be rolled again to form an additional hemostat if desired in some embodiments of the present disclosure. In another embodiment of the present disclosure, instead of rolling the gel into a sheet, the foaming action gel may be poured into a mold, preferably non-stick, and the sterile applicator stick or tube or wire may then be inserted making a “popsicle.” Regardless of what method is utilized for drying, once dry, the hemostat may be placed in a sterile bag capable of gas, radiation or heat sterilization. The sterile bag or package should be easy to open for fast use in treatment. The construction of the chemically foaming hemostat may be in the form of a non-gel powder or a gel or a non-gel liquid.

As previously described, bleeding may be controlled and/or blood clotting may be accelerated through inclusion of one or more pharmaceuticals or compounds as part of the expanding, flowing foaming gel. Clotting agents can include, but are not limited to, 0.2% to 14.5% tranexemic acid with 0.3% to 5% chitosan in a base of 5% to 45% alginic acid calcium salt derivatives or other effective compounds in desired concentrations. While the clotting agents may be pharmaceutical grade, it should be appreciated that other non-pharmaceutical clotting agents may be used in addition to or in place of pharmaceutical clotting agents without departing from the present disclosure.

Infection may be controlled through inclusion of one or more pharmaceuticals and/or compounds, such as nano-sized silver particles. Nano-sized silver particles, usually having a size of under 30 nanometers generally applied at approximately 800 ppm or more, may pass through cell membranes of viruses, bacteria, molds and funguses and kill them by combining with the oxygen in the cell suffocating it and killing the contaminant. Accordingly, these nano-sized silver particles may mitigate infection by killing/controlling/decreasing bacteria, viruses, mold, spores and fungi, and other pathogens upon contact. Further, the remaining nano-sized silver particles have a long-term residual anti-infection/protective effect on tissues that remains long after the hemostat foam carrier medium and other antiseptic agents have dried and dissipated. While embodiments of the present disclosure have disclosed use of nano-sized silver particles to control infection, other pharmaceuticals and/or compounds may be utilized in addition to or in place of nano-sized silver particles to treat infection without departing from the present disclosure.

Shock control may be accomplished by inclusion of one or more pharmaceuticals or compounds, such as 0.025% to 2.5% epinephrine or similar or other products, as part of a foaming gel or powder or liquid.

Pain control may be accomplished by inclusion of one or more pharmaceuticals or compounds, such as codeine or other narcotics, liquid lidocaine (2%-10%) or a lidocaine/prilocaine combination (emla); however, the type and amount of pain control agent may vary depending on the patient.

Sepsis control may be accomplished by inclusion of one or more pharmaceuticals or compounds known to control sepsis, including but not limited to antiseptics and antibiotics.

A chemically foaming hemostat according to an embodiment of the present disclosure may include a foaming gel or liquid or powder comprised of approximately 0.2% to 14.5% tranexemic acid with approximately 0.3% to 5% chitosan in a base of approximately 5% to 45% alginic acid calcium salt derivatives mixed with approximately 0.025% to 2.5% epinephrine and packaged in a dried gel, liquid gel, solid or powder wound “plug” to keep the pharmaceuticals and compounds in suspension to prevent them from settling or separating out. However, other compositions may be utilized without departing from the present disclosure.

As previously discussed, a hemostat according to an embodiment of the present disclosure may include an insertion mechanism taking the form of a sterile stick or tube or wire that may be shaped in a manner to facilitate insertion into a wound, particularly deep wounds, puncture wounds, and internal injuries. A plug formed of the foaming gel and one or more compounds, whether taking the form of a solid material, a sponge or a sponge-like material, a liquid, a gel, or a dried gel, or a powder may be affixed to the stick or insertion device, and then pushed into the bottom/deepest portion of a wound. The stick may be removed after the contents begin to foam. However, it should be appreciated that leaving the stick in the wound after foaming begins to occur is not believed to have short-term harmful effects and may even be desirable in some embodiments of the present disclosure. It should be appreciated that the plug may be dried into a shape suitable for insertion into a wound, such as sheets that may be rolled together in a tampon-like arrangement. Moving or transportation of the patient may cause bleeding to start again. After application of a foaming action hemostat according to an embodiment of the present disclosure, if the wound begins to bleed again, one or more additional foaming action hemostats may be applied until bleeding is stopped. This process may be repeated without harm, if undesired bleeding begins again.

In some embodiments of the present disclosure, there may be a cover shield tube forming at least part of the insertion mechanism for the hemostat. The stick or tube or wire may hold the hemostat plug in place in the wound as the cover shield tube is withdrawn. The plug portion of the hemostat that contacts the wound generally may assume a rounded shape to facilitate insertion into the wound. However, it may assume other shapes without departing from the present disclosure. It also should be appreciated that the hemostat may be flexible, not brittle, in nature to allow it to follow a circuitous path into the wound during insertion.

In another embodiment of the present disclosure, the hemostat may be a solid and/or a liquid or a powder introduced into a wound using a syringe or injection mechanism containing an insertion tube or a similar type applicator that may assist in insertion of the contents of the hemostat into a wound's deepest part. Upon insertion, the syringe (or a funnel, if used) and insertion tube may be removed. It should be appreciated that the hemostat liquid may be an un-dried liquid gel that may be injected into the wound with a syringe until the wound begins foaming.

The hemostat may include additional tubing, such as an infusion tube, that may allow more or additional pharmaceuticals and/or compounds to be injected into or through the tube into a wound before the insertion mechanism is withdrawn. Additionally, or alternatively, introduction of tubing may allow for suction/withdrawal/removal/addition of fluid from the bottom of the wound. It should be appreciated that more than one piece of tubing may be inserted into the wound initially along with the hemostat gel, and left in place for addition of pharmaceuticals and/or other compounds, to withdraw fluids, take temperature, or other data readings, or heat/cool/circulate fluids or gases associated with the wound without departing from the present disclosure.

The foaming action hemostat also may include one or more sensors, detectors, wiring or even fiber optics so that the wound may be monitored and data may be collected. For example, when a gel is rolled around or cast onto a stick to form the hemostat, other wires and/or sensors may be introduced along with the gel so that they can remain in the wound for monitoring (i.e., temperature, pH, pressure), sampling, evacuation, and addition of medications, among other applications. They may then be removed electively at some later time.

In some embodiments of the present disclosure, the foaming action hemostat may be formed as a powder (as opposed to a gel) that may be poured, sprinkled or dusted onto a wound. Additionally, or alternatively, the foaming action hemostat may be supplied as a liquid to be dripped onto or squirted or sprayed into a wound, and may be used with a syringe having a dispensing tube. In a further embodiment of the present disclosure, the hemostat may be configured as different sized sheets or patches that may be applied topically to a non-punctured surface wound site such as an abrasion or burn or packed into larger wound sites to address bleeding. Similarly, a liquid or gel forming part of the hemostat may be placed as dots or shapes on a wound dressing, a sterile pad, or a bandage to stop blood loss while applying pharmaceuticals and/or other compounds to the wound. The foaming action hemostat may have a piercing or cutting device applied to the end of the stick or tube or wire to facilitate cutting through tissues to insert the hemostat into the depth of the wound, or in the case of internal injuries, to facilitate cutting through covering tissues in order for the hemostat to reach the area that is bleeding.

In some embodiments of the present disclosure, a foaming action hemostat may contain a radiation or x-ray marker or dyes. This radiation marker may allow for easier identification of the wound depth, path and position when the hemostat is introduced into a wound. Further, it may make it easier for medical personnel to find, identify and/or remove the plug or foaming gel portion of the hemostat from the wound at a later time. The marker may be blended into the gel of the hemostat; however, it also may be attached to the foaming action hemostat (i.e., may be formed separately from the gel). While the marker has been described as using radiation, it should be appreciated that detection through a marker may be done using x-ray, infrared, ultraviolet radiation, “radioactivity” or other detection mechanisms without departing from the present disclosure. There also may be embodiments of the present disclosure where compounds may be included within the hemostat to dye the foam in a color that may make eventual removal/flushing of the gel/liquid/powder easier.

In some embodiments of the present disclosure, a field dressing may be placed over the wound after the hemostat has stopped the bleeding; however, the field dressing will not be placed inside the wound being treated using the foaming action pharmaceutical hemostat. For example, when the gel of a hemostat foams, the stick portion may be removed, and a field dressing may be applied. It may be desirable that the field dressing has a non-stick surface so it is less likely to clot onto the wound. It should be appreciated that an active foaming action may occur in the wound, and applying a field dressing to the wound may seal the foaming action. It should further be appreciated that foaming may expand within the wound until it creates a pressure in the wound. It should also be appreciated that the foam may expand and flow to permeate pharmaceuticals and non-pharmaceutical ingredients among each and every tissue throughout the wound in embodiments of the present disclosure. Such field dressing may be included in the hemostat packaging. Other wound care accessories may be included in the hemostat packaging including, but not limited to, gloves, sterile masks, tweezers, mechanical hemostats, flashlight, flares, suture equipment, bandages and dressings, “butterfly” bandages, tape, scissors, scalpels, packages or syringes of medicines and pharmaceuticals, medications, writing instruments (such as a laundry market or pen or pencil), a tag or label to write on, tourniquets, and other such items as may be desired to be packaged with the hemostat to include other pharmaceuticals, chemicals, suture kits and other wound care necessities.

While certain pharmaceuticals and compounds have been described herein, it should be appreciated that the number and type of pharmaceuticals or other compounds included as part of the foaming action hemostat may be altered depending on the effect or use desired. For example, different pain medications or other therapeutic or non-therapeutic compounds may be included in the foaming action hemostat depending on the treatment desired. It also should be appreciated that the quantities may change, and uses/applications other than treatment of bleeding, sepsis, infection, shock and pain and other symptoms may be contemplated though not specifically mentioned herein. It should be appreciated that the dose effectiveness of pharmaceuticals and/or other compounds introduced into a wound may remain constant throughout use. It should further be appreciated that blockage of harmful contaminants from the surface of the wound may continue over an extended period of time, but may be removed through washing/scrubbing, flushing and/or suction techniques in some embodiments of the present disclosure.

In some embodiments of the present disclosure, the foaming action hemostat may be applied once to a wound for long-term residual blocking of harmful contaminants; however, there may be other embodiments of the present disclosure where more than one application may be needed/desired. For example, in the case of a large wound, more than one foaming action hemostats may be inserted over time or even may be applied to the wound simultaneously. It should be appreciated that the size of a hemostat according to embodiments of the present disclosure may change depending on the type of wound being treated. For example, a hemostat may be provided in a very small diameter size for application into small diameter puncture wounds.

It should be appreciated that the foaming action hemostat may be used for treatment of areas other than wounds without departing from the present disclosure. Further, while the foaming action hemostat has been described with respect to use on humans, it should be appreciated that it may be used on non-humans without departing from the present disclosure.

It should be appreciated that a foaming action hemostat, according to embodiments of the present disclosure, may be sterilized (such as with gas, radiation or radioactivity) and placed within sealed packaging so that it may have a lengthy shelf life. This may be particularly useful in certain situations, such as on the battlefield or in extreme conditions of moisture, heat, cold, and/or dirt. Even if jostled within the packaging, the pliable nature of the foaming action hemostat may tolerate some degree of crushing and impact without losing its effectiveness. The packaging should be easy to open so that it can be accessed quickly in an emergency situation to improve a patient's recovery or survival time. It also may be desirable that the packaging holding the hemostat may be lightweight and weatherproof so that field personnel may easily carry quantities of hemostats according to embodiments of the present disclosure.

Further, a hemostat according to embodiments of the present disclosure may be easy to use so that a person having little-to-no medical training and expertise may still properly apply the hemostat to a range and sizes and types of wounds. The packaging may include instructions for use in several languages and drawings to instruct its use to the illiterate.

Although the present disclosure and its advantages have been described in detail, it should be understood that various changes, substitutions and alterations can be made herein without departing from the spirit and scope of the disclosure as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

1. A foaming action pharmaceutical hemostat, comprising:
at least one fully dissolvable sheet of material provided to form the foaming action pharmaceutical hemostat, wherein the foaming action pharmaceutical hemostat is arranged in a wound, foams vigorously in the wound, fully dissolves in the wound, and leaves no harmful residue in the wound.

2. The foaming action pharmaceutical hemostat of claim 1, wherein the foaming action pharmaceutical hemostat is compressed and remains compressed in the wound until fully dissolved.

3. The foaming action hemostat of claim 1, wherein the at least one fully dissolvable sheet of material is pre-foamed and dries with gas bubbles to resemble a sponge structure.

4. The foaming action hemostat of claim 1, wherein the at least one fully dissolvable sheet of material is provided in the form of a field dressing.

5. The foaming action hemostat of claim 1, wherein, upon foaming action, the at least one fully dissolvable sheet of material permeate pharmaceuticals throughout the wound.

6. The foaming action hemostat of claim 1 further comprising:
an insertion mechanism, wherein the insertion mechanism is a sterile stick or tube or wire.

7. The foaming action hemostat of claim 6, the insertion mechanism further comprising:
a cover shield tube covering the sterile stick or tube or wire, wherein upon insertion of the foaming action pharmaceutical hemostat into the wound, the sterile stick or tube or wire holds the foaming action pharmaceutical hemostat in place and the cover shield tube is withdrawn.

8. The foaming action hemostat of claim 1 further comprising:
a radiation or x-ray marker or other material that can be detected and will disclose the location of the material throughout the wound.

9. The foaming action hemostat of claim 1 further comprising:
one or more sensors, detectors or wires to monitor the wound.

10. The foaming action hemostat of claim 1 further comprising:
one or more tubes to evacuate and introduce a solid or liquid into the wound.

11. A foaming action hemostat comprising:
a fully dissolvable sheet or piece of material; and
a foaming gel comprising:
a first mixture formed from mixing an anhydrous gelling agent with a sterile anhydrous liquid; and
a foaming agent and a pharmaceutical-type clotting agent added to the first mixture and blended to form the foaming gel, wherein the foaming gel is arranged to aggressively foam, expand throughout a wound, until bleeding in the wound abates,
wherein the foaming action hemostat is arranged in the wound, fully dissolves in the wound, and leaves no harmful residue in the wound.

12. The foaming action hemostat of claim 11, the foaming gel further comprising:
one or more additional compounds selected from the group comprising: shock mitigation agents, antibiotics, nano-silver particles, pain relieving agents, and anti-sepsis agents.

13. The foaming action hemostat of claim 11, wherein a sterile stick or wire is provided to arrange the foaming action hemostat in the wound.

14. The foaming action hemostat of claim 11, wherein the foaming action hemostat remains compressed in the wound until the foaming action hemostat fully dissolves.

15. The foaming action hemostat of claim 11, wherein the foaming action hemostat is water and therefore blood-soluble.

16. The foaming action hemostat of claim 11, wherein the foaming action hemostat automatically begins to dissolve and foam immediately after insertion into the wound and contacting with the water in the blood.

17. The foaming action hemostat of claim 11 wherein the foaming action hemostat fully dissolves and stops foaming after a predetermined period of time.

18. The foaming action hemostat of claim 11 wherein the foaming action hemostat permeates pharmaceuticals and other ingredients throughout the wound.

19. A hemostatic apparatus, the apparatus comprising:
a fully dissolvable foaming action hemostat including a foaming agent, wherein the fully dissolvable foaming action hemostat is introduced into a wound and fully dissolves in the wound after foaming; and
a sterile stick or wire provided to introduce the foaming action hemostat into the wound,
wherein the foaming action hemostat takes a form selected from the group comprising:
a sheet, a shape, a liquid, a powder, or small cubes, and
wherein upon contact with the water in the blood, the foaming agent activates to foam in the wound and bleeding associated with the wound is controlled or stops.

20. The apparatus of claim 19, wherein the foaming agent is arranged to impregnate the wound with one or more compounds selected from the group comprising:
clotting agents, shock mitigation agents, antibiotics, nano-silver particles, pain relieving agents, and anti-sepsis agents, wherein activation of the foaming agent in the wound introduces the one or more compounds into the wound.
